(19) United States
(12) Patent Application Publication
(10) Pub. No.: US 2010/0268339 A1
(43) Pub. Date: Oct. 21, 2010

(54) Intervertebral Spinal Implant and Method of Making the Same

(76) Inventors: Theodore I. Malinin, Key Biscayne, FL (US); H. Thomas Temple, Miami, FL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(21) Appl. No.: 12/823,811

(22) Filed: Jun. 25, 2010

Related U.S. Application Data
(63) Continuation-in-part of application No. 11/775,656, filed on Jul. 10, 2007.

(51) Int. Cl. A61F 2/44

(52) U.S. Cl. 623/17.11

(57) ABSTRACT

An invertebral implant for replacing a damage invertebral disk within the spinal column can include a generally flat body having opposing surfaces and peripheral wall or surface that extends between the opposing surface. Protruding outwardly from the peripheral wall can be a shelf-like flange that has a reduced thickness compared to the thickness of the main body of the implant. The flange provides an object or structure that the surgeon can grasp with forceps during insertion between adjacent vertebrae. Because the protruding nature and reduced thickness of the flange, both the flange and the forceps placed thereon can fit within or adjacent to the intervertebral space between the adjacent vertebrae without interfering with orientation or placement of the main body between the vertebrae. Hence, the flange may simplify the surgical insertion procedure.

### CROSS-REFERENCE TO RELATED APPLICATIONS

This patent application is a continuation-in-part of copending U.S. patent application Ser. No. 11/775,656, filed Jul. 10, 2007.

### BACKGROUND OF THE INVENTION

In humans and other vertebrate animals, the spinal column is made of individual bones or vertebrae that are aligned together and extend along the center of an individual's back. Importantly, the spinal column provides a protective channel for the spinal cord of the central nervous system and supports an individual's weight and posture while enabling a wide range of motion of the upper body. The vertebrate are movably joined at facet joints and, in humans in particular, can be arranged in regions including the cervical region corresponding to the neck, the thoracic region corresponding to the chest, and the lumbar region corresponding to the lower back. The arrangement of vertebrae within the regions can provide the familiar curves and arches of the spinal column. To enable bending, twisting and rotating of the upper body, the individual vertebrae are spaced apart by intervertebral disks. The intervertebral disks are made of a tough, fibrous connective tissue that rings around and surrounds a thick, jelly-like material at the center of each disk. The disks act to dampen shock transmitted along the spinal column and to enable motion.

Intervertebral disks may become damaged or degenerate overtime, due to disease, or due to abrupt injury such that it may become medically necessary or beneficial to surgically remove the damaged disk. To maintain the intervertebral spacing between two adjacent vertebrae from which a disk has been removed, it is known to insert spinal or intervertebral implants into the space. The intervertebral implant preferably promotes bone growth to fuse the adjacent vertebrae across the disk space. A variety of materials, sizes, shapes, and insertion techniques have been suggested for providing and inserting intervertebral implants. For example, it is well known to shape the implants as cylindrical dowels that can be inserted between the vertebrae. In some instances, the implant can be formed of a biocompatible material such as metal or ceramic or can be formed from actual bone tissue harvested from a donor bone. Desirably, the material, size and shape of the implant are selected for ease of implantation, maintenance of the proper spinal curvature, and to provide the necessary biomechanical strength to support the spinal column.

In some instances, screws, braces or fixtures can be utilized to maintain alignment of the spinal column and implant during recovery and fusion of the adjacent vertebrae. In other instances, it may be desirable to incorporate osteogenic material with the intervertebral insert to promote bone tissue growth and fusion of the adjacent columns. Accordingly, there exists a need for an intervertebral spinal implant that can maintain the intervertebral space between and enable rapid fusion of adjacent vertebrae. There exist a further need for a intervertebral implant that is biologically active and biomechanically strong and that can maintain and support the existing curvature of the spinal column. Additionally, the intervertebral implant should remain stable and not be prone to slippage.

### BRIEF SUMMARY OF THE INVENTION

The invention provides an intervertebral spinal implant for maintaining intervertebral spacing between and promoting the fusion together of two adjacent vertebrae. In an aspect, the intervertebral implant can have a generally flat body with a first surface and an opposing second surface that is sized and shaped for insertion into the intervertebral space. Disposed into the body can be at least one aperture that can be formed to receive osteogenic or similar medicinal material that promotes bone growth between the vertebrate to fuse those vertebrate together. To optimize retention of the osteogenic material within the body during manipulation of the implant, the aperture in some embodiments can be disposed on a non-perpendicular angle into the first surface of the body. In other embodiments, the aperture can taper or be conically shaped as it extends from the first surface toward the second surface of the body. The tapering of the aperture can be in addition to or besides disposing the apertures on non-perpendicular angles. Another advantage of disposing the osteogenic material receiving aperture on a non-perpendicular angle or on a taper is that the material will tend not to shake or fall loose from the aperture. Another advantage is that the non-perpendicular or tapered apertures can accommodate more osteogenic material.

In another aspect of the invention, an intervertebral implant having a flat body with first and second opposing surfaces can have disposed into at least one surface a plurality of grooves. The grooves can have any suitable shape or pattern, but preferably have a gull-wing shape. To provide the gull-wing shape, the grooves can have a first curve and a second curve that intersect together approximately mid-width of the implant. The gull-wing shaped grooves can retain osteogenic or other medicinal material and can allow for ingrowth of the host bone. In various embodiments, the intervertebral implant can have gull-wing shaped grooves on both the first and second surface and further can include one or more osteogenic material receiving apertures of the above described kind. Another advantage of disposing the gull-wing shaped grooves across a surface of the implant is that grooves provide traction where the implant surface meets the vertebrae thereby preventing slipping or movement of the implant.

In another aspect of the invention, an intervertebral implant having a flat body and first and second opposing surfaces can be formed from the elongated diaphysis or shaft portion of a long donor bone. To form the implant, a plurality of outlines, each of the first surface, are cut or otherwise disposed directly into the outer surface of the bone tissue such that the plurality of outlines are arranged axially along the diaphysis. Accordingly, one surface of the implant corresponds to the outer surface of the diaphysis of the donor bone. This is in contrast to prior art methods, in which allografts or spinal implants are typically formed by disposing cuts perpendicularly into the diaphysis. An advantage of preparing the implants by cutting into the disphysis parallel rather than perpendicular to its long axis is to conserve donor bone by enabling larger and more implants to be formed from a given bone.

In another aspect of the invention, the intervertebral implant can have a generally flat body generally shaped overall as a question mark. The question-mark shape can be provided by having a peripheral surface of the body include a straight first edge, a curved second edge extending away from the first edge, and a cutout formed into the first edge. In various embodiments, the cutout can receive osteogenic or other medicinal material. An advantage of forming the implant with a question-mark shape is that such a shape helps to fill the entire intervertebral space.

To simplify grasping and manipulating the intervertebral implant with forceps or the like, in a further aspect of the invention, the implant can include one or more shelf-like flanges that protrude outwardly from the peripheral surface of the implant. The flange provides a flat, protruding block or boss that can be grasped with forceps or clamps during the surgical insertion procedure. Moreover, the flange can have a reduced thickness compared to the main body of the implant such that, when the implant is sandwiched between two adjacent vertebrae in the intervertebral space, the flange is freely suspended between and separated from the upper and lower vertebrae. Thus, the flange is still accessible and can be grasped by inserting the forceps into or proximate to the intervertebral space. Also disclosed is a method of surgically inserting the implant into the spinal column by manipulating the flange.

Accordingly, an advantage of the inventive intervertebral implant is that it provides strong biomechanical support to the spinal column. Another advantage is that the intervertebral implant can retain osteogenic material for promoting fusion of adjacent vertebrae. A related advantage is that the intervertebral implant can include curved grooves of a specific shape to prevent slipping of the implant from between adjacent vertebrae. Yet another advantage is that the intervertebral implant can be shaped to promote and maintain the lordotic curve of the lumbar region in the spine. Another advantage is that inclusion of the flange on a spinal implant facilitates grasping and manipulating the implant with forceps. These and related advantages and features of the invention will become apparent upon review of the following drawing and

### BRIEF DESCRIPTION OF THE DRAWINGS

FIG. 1 is a top perspective view of an intervertebral spinal implant having a general “D”-shaped outline and a plurality of apertures disposed on a non-perpendicular angle therein.

FIG. 2 is a side elevational view of the intervertebral implant of FIG. 1 showing the non-perpendicular angle at which the apertures are disposed there through.

FIG. 3 is a schematic diagram illustrating one method of inserting the intervertebral implant into an intervertebral space of the spinal column.

FIG. 4 is a top plane view of another embodiment of an intervertebral implant having a “D”-shaped outline and a plurality of tapering apertures disposed on a non-perpendicular angle therein.

FIG. 5 is a side elevational view of the intervertebral implant illustrating the non-perpendicular angle that the tapered shape apertures are disposed along.

FIG. 6 is a bottom plan view of the intervertebral implant of FIG. 4.

FIG. 7 is a top plane view of another embodiment of an intervertebral implant having an elongated “D”-shape having a plurality of aperture disposed therein on various different non-perpendicular angles.

FIG. 8 is a top perspective view of another embodiment of an intervertebral implant having a plurality of non-perpendicular apertures and plurality of grooves disposed into a surface thereof, the grooves each having a gull-wing shape, the apertures and grooves retaining an osteogeneric material.

FIG. 9 is a perspective view of a diaphysis or shaft of an elongated donor bone having the outline of a plurality of intervertebral implants disposed therein in accordance with an aspect of the invention.

FIG. 10 is a top perspective view of an intervertebral implant having a question-mark shape.

FIG. 11 is a perspective view of another embodiment of the intervertebral spinal implant having a protruding flange for grasping with a pair of forceps or the like.

FIG. 12 is a side elevational view of the intervertebral spinal implant illustrated in FIG. 11 illustrating the thickness of the implant with respect to the thickness of the flange.

FIG. 13 is a schematic diagram illustrating a method of inserting the intervertebral implant into an intervertebral space by grasping the flange with a pair of forceps.

FIG. 14 is a schematic diagram illustrating the intervertebral spinal implant situated in the intervertebral space between adjacent vertebrae.

FIG. 15 is a top plan view of another embodiment of an intervertebral spinal implant having first and second flanges.

FIG. 16 is a top plan view of another embodiment of an intervertebral spinal implant having a posterior flange extending from the rear edge of the implant.

FIG. 17 is a top plan view of another embodiment of an intervertebral spinal implant having three flanges.

FIG. 18 is a perspective view of another embodiment of an intervertebral spinal implant having a flange formed separately from and attached to the main body of the implant.

FIG. 19 is a schematic diagram showing another embodiment of the intervertebral spinal implant having a tongue and groove design with two implants positioned laterally side-by-side in the intervertebral space.

FIG. 20 is a schematic diagram showing another embodiment of the intervertebral spinal implant having a tongue and groove design with two implants stacked vertically in the intervertebral space.

FIG. 21 is a perspective view of another embodiment of an intervertebral spinal implant having a central aperture and a plurality of micro-perforations.

FIG. 22 is a top plan view of the spinal implant of FIG. 21 further showing the corrugated lateral edges of the implant.

### DETAILED DESCRIPTION OF THE EMBODIMENTS

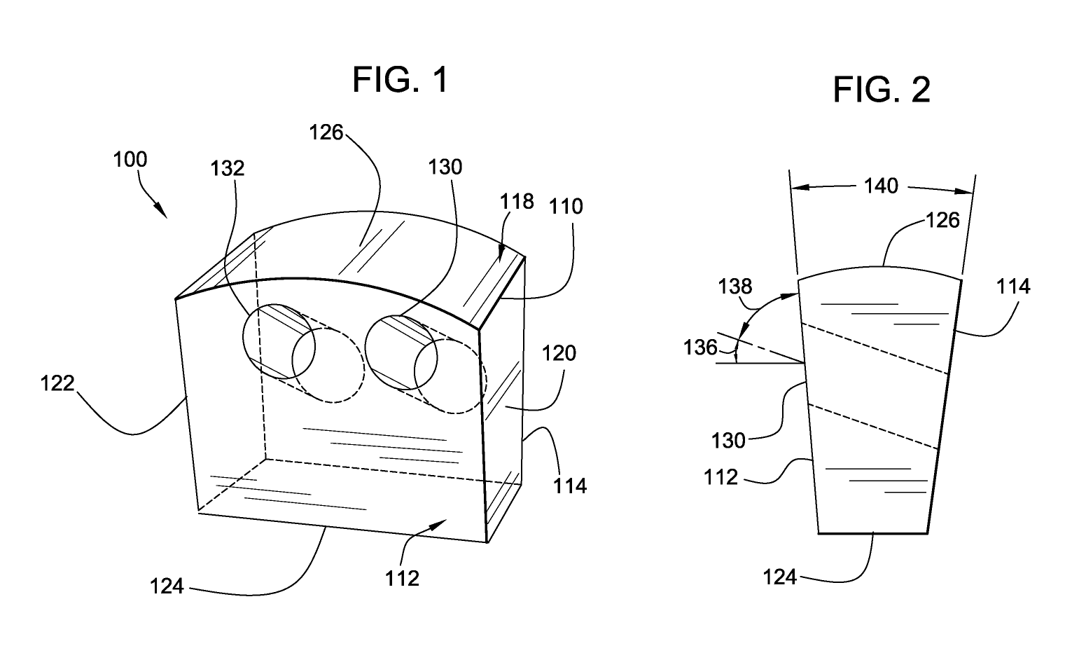

Now referring to the drawings, wherein like numbers refer to like elements, there is illustrated in FIGS. 1 and 2 an intervertebral implant 100 that can replace a damaged or ruptured intervertebral disk within the spinal column. The generally solid implant 100 can have a block-like shape including a generally flat body 110 including a first surface 112, an opposing second surface 114 and a peripheral surface 118 extending between the opposed first and second surfaces. In the illustrated embodiment, the intervertebral implant 100 can have a “D” shape in which the peripheral surface 118 further includes a first straight lateral edge 120, a second straight lateral edge 122 parallel to and spaced apart from the first lateral edge, and a third straight edge 124 extending between the first and second lateral edges. The peripheral surface further includes a curved edge 126 extending between the first and second edges 120, 122 and directed away from the third edge 124. As can be appreciated, the four edges of the peripheral surface generally provide the block-like shape to the intervertebral implant 100.

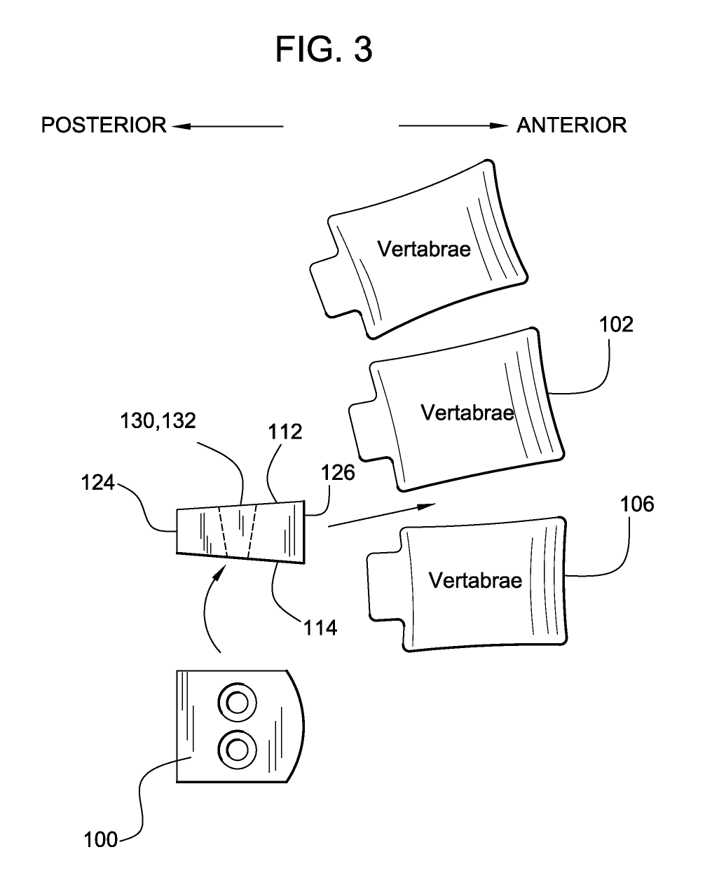

Replacement of a disk with the intervertebral implant 100 can be illustrated with reference to FIG. 3. First, the implant 100 is aligned between adjacent vertebrae including a upper vertebrae 102 and a corresponding lower vertebrae 106. The vertebrae can be separated by appropriate manipulation of the spinal column and the damaged intervertebral disk removed. The implant 100 can be aligned so that the curved fourth edge 126 is directed towards the anterior as indicated. Moreover, the upper surface 112 of the implant is oriented toward the upper vertebrae 104 and the lower surface 114 is oriented toward the lower vertebrae 106. The implant 100 is then inserted into the intervertebral space between the adjacent vertebrae and the upper and lower vertebrae can come to rest adjacent the respective upper and lower surfaces 112, 114. Preferably, the intervertebral implant 100 can assume a majority of the intervertebral space vacated by the removed disk. Because the implant is substantially solid, it can provide sufficient biomechanical strength for supporting the spinal column. While FIG. 3 illustrates insertion from the posterior direction, in other embodiments, insertion can occur from the anterior or other suitable direction.

Referring back to FIGS. 1 and 2, the intervertebral implant 100 can have any size suitably selected for the particular disk the implant is intended to replace. By way of example only, the implant can have an average width between the first lateral edge 120 and the second lateral edge of about 3.0 cm. Moreover, the implant 100 can have an average length between the third straight edge 124 and the apex of the fourth curved edge 126 of between about 1.3 cm to about 2.5 cm. The thickness of the implant 100 between the first surface 112 and the second surface 114 can be between about 0.5 cm and 2.0 cm. In other embodiments, these dimensions can be selected to traverse the majority of the length and width of the intervertebral space such that surface area of contact between upper and lower vertebrae is about 68 square cm and can be 200 square cm or more.

The material of the intervertebral implant 100 can be selected from any suitable biocompatible material having the desired biomechanical strength, immune acceptance and toxicity characteristics. For example, the material can be selected from a biologically compatible metal such as titanium, cobalt or chrome steel, gold alloys, stainless steel or similar metals. In other embodiments, the material can be selected from a synthetic, biologically active or bio-absorbable material such as calcium, sulfate, polyglycolic acid, hydroxyapatite, porous ceramics, apatitic bone cement, calcium phosphate, hydroxyproline, hydroxyapatite cement, and methylmetacrylate. In certain embodiments, the implant material can be selected from bio-active or bio-inactive bone tissue. For example, the bone tissue can be primarily cortical tissue such as typically found on the hard, solid outer surface of a donor bone. The bone tissue can also be primarily spongy cancellous bone typically found in the interior of thicker bones. When used in intervertebral implants, cortical bone may be desirable for its biomechanical strength properties but cancellous bone may be desirable for its ability to promote vascularization and new bone growth to fuse the adjacent vertebrae together. Accordingly, to increase biomechanical strength, the implant can be made from 90% cortical bone while to promote bone growth, the implant can be made from about 60% to 98% cancellous bone. When bone tissue is taken from a donor bone to form the intervertebral implant, the donor bone can be selected such that the resulting intervertebral implant can be an allograft (same animal species) or a xenograft (different animal species).

To address the above paradox, the intervertebral implant 100 can be configured to have an implant body 110 of relatively harder material and to retain or include a bioactive osteogenic material or similar medicinal material. To carry the osteogenic material, the implant body 110 can have a first aperture 130 and a second aperture 132 disposed into the first surface 112 and directed toward the second surface 114. The apertures 130, 132 may or may not traverse the entire thickness of the implant body 110. Moreover, in the illustrated embodiment, the apertures can be circular in cross-section but in other embodiment may have different shapes. The osteogenic or medicinal material can be placed or packed into the apertures 130, 132 prior to insertion of the implant into the intervertebral space. As can be appreciated with respect to FIG. 3, because the apertures 130, 132 are disposed through the first and second surfaces 112, 114 that are placed physically adjacent the upper and lower vertebrae, the osteogenic material can contact the vertebrae to promote bone grown and fusion.

The osteogenic material can be selected from any suitable material that helps promote bone growth and thereby speed fusion of adjacent vertebrae. For example, the osteogenic material can be selected from non-de-mineralized particular bone material, de-mineralized bone matrix, partially de-mineralized bone material, partially de-calcified bone material, AAA bone graft, or osteogenic growth factors including BMP. Moreover, the osteogenic material can be provided as a particulate, a jelly, a paste or a putty.

To optimize retention of the osteogenic and/or medicinal material in the aperture during handling and insertion of the implant 100, the apertures 130, 132 can be disposed on a non-perpendicular angle into the first surface 112 and towards the opposing second surface 114. Specifically, as best illustrated in FIG. 2, the axis line of the aperture 130 can be disposed on an angle 136 with respect to an imaginary line extending normally from the plane of the first surface 112. Angle 136 can be any suitable angle, for example, about 20°. Accordingly, the aperture 130 is disposed on a complementary angle with respect to the plane of the first surface 112 itself. An advantage of the non-perpendicular apertures 130, 132 is that they can better retain the osteogenic material by preventing the material from shaking loose or falling out of the insert and for accommodating a larger volume of osteogenic material. For example, non-perpendicular apertures have more exposed surface area to frictionally contact the osteogenic material than would perpendicular apertures. Additionally, the non-perpendicular apertures 130, 132 ensure that the retained material is not directly acted upon by gravity when the implant 100 is laid on either surface 112, 114 but is instead supported by the corresponding material of the body 110 delineating the channel of the apertures. Another advantage of disposing the apertures 130, 132 on a non-perpendicular angle is that the apertures define a greater volume through the body 110 and can therefore retain more osteogenic material.

Continuing to refer to FIG. 2, the opposing first and second surfaces 112, 114 of the intervertebral implant 100 need not be parallel with each other but instead can diverge on a given angle 140 from each other with respect to the straight third edge 124. The angle 140 is preferably selected so that the overall shape of the intervertebral implant 100 remains substantially flat. For example, the angle 140 can be about 9°. An advantage of angling the first and second surfaces 112, 114 with respect to each other can be appreciated with respect to FIG. 3. When the intervertebral implant 100 is inserted into the intervertebral space with the resulting thicker fourth edge 126 oriented toward the anterior of the spinal column and the relatively thinner third edge 124 oriented toward the posterior, it will be appreciated that the adjacent vertebrae 102, 106 are likewise maintained at an angle with respect to each other within the spinal column. Angling the vertebrae helps maintain the lordotic curve of the spinal column in the lumbar region of the lower back. Otherwise, loss of the lordotic curve may result in imbalance problems occurring to patient, pain, loss of the motion, and improper fusing of the adjacent vertebrae.

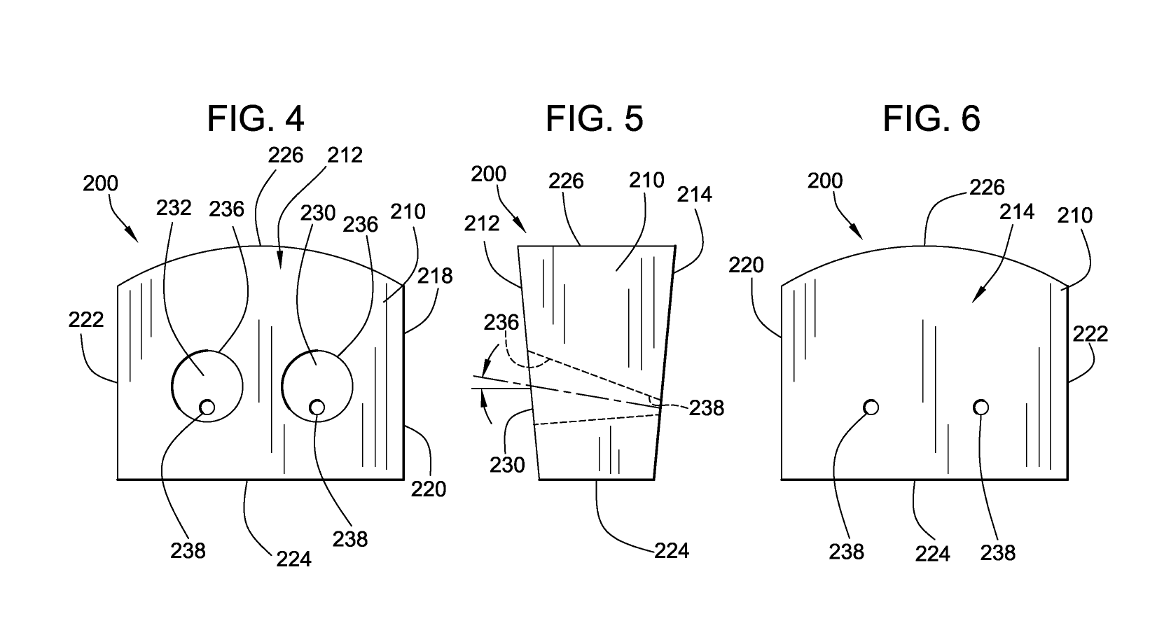

Referring now to FIGS. 4-6, there is illustrated another embodiment of an intervertebral implant 200 for replacing a damaged disk between two adjacent vertebrae. The illustrated intervertebral implant 200 again has a relatively solid implant body 210 having a block-like shape with a first surface 212, an opposing second surface 214, and a peripheral surface 218 extending there between. In the illustrated embodiment, the peripheral surface 218 again outlines or provides a “D” shape to the implant body 210. Particularly, the peripheral surface 218 can include a first straight lateral edge 220, a parallel and spaced apart second straight lateral edge 222, and a third straight edge 224 extending between the first and second laterals edges. The peripheral edge 218 also includes a fourth curved edge 226 extending between the first and second straight edge 220, 222. The fourth curved edge 226 is spaced apart and curves away from the third straight edge 224. Of course, in other embodiments, the implant body 210 can have other suitable shapes. Additionally, the intervertebral implant 200 can have dimensions corresponding to those provided above.

To retain the osteogenic or medicinal material, the intervertebral implant 200 can include a first aperture 230 and a second aperture disposed into the first surface 212 and directed toward the second surface 214. While in the illustrated embodiment, the apertures are disposed entirely through the implant body 210, it will be appreciated that in other embodiments, the apertures may terminate prior to the second surface 214. To optimize retention, the apertures 230, 232 can have a tapered or conical shape as they are disposed through the implant body 210 from the first surface 212 toward the second surface 214. Specifically, the circular apertures 230, 232 can form a larger diameter hole 236 proximate the first surface 212 and a smaller diameter hole proximate 238 the second surface 214. Tapering the apertures cause more surface area of the implant body 210 to frictionally contact the osteogenic material, thereby preventing the material from shaking or falling loose of the intervertebral implant 200. Additionally, the smaller diameter hole 238 restricts the osteogenic material from passing out the apertures 230, 232 via the second surface 214. In another embodiment, instead of tapering the aperture, the aperture can be formed as a counterbore having a first section of a larger diameter disposed into the first surface and a second section of a smaller diameter disposed into the second surface. Accordingly, in the present embodiment, the intervertebral implant 200 is inserted into the intervertebral space such that the second surface 214 is oriented toward the lower vertebrae.

To further improve osteogenic material retention, the tapered aperture 230, 232 can also be disposed into the first surface 212 on a non-perpendicular angle. Specifically, as illustrated with respect to FIG. 5, the axis line of the aperture 230 is offset with respect to an imaginary line extending perpendicularly from the plane of the first surface 212 by an angle 240. Angle 240 can be any given angle including, for example, 20°. Disposing the apertures 230, 232 on a non-perpendicular angle can realize the benefits mentioned above with respect to FIGS. 1 and 2.

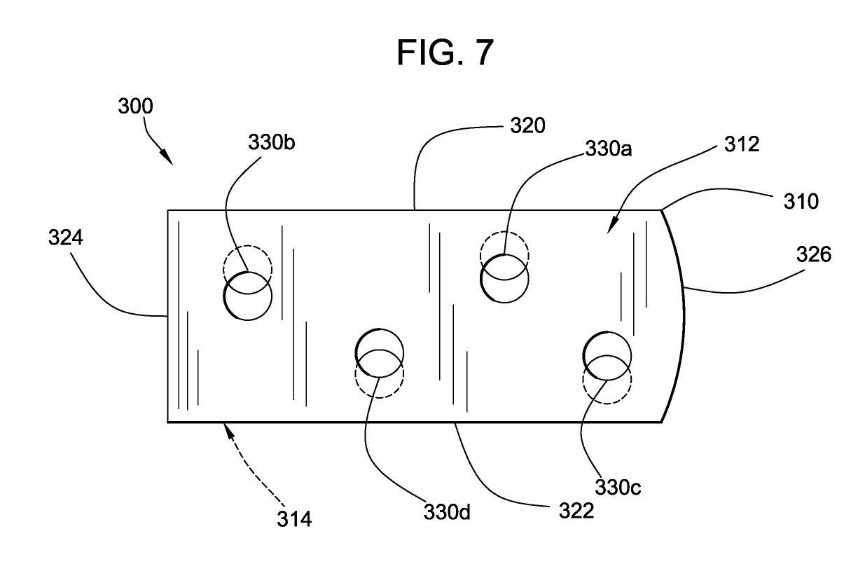

Referring now to FIG. 7, there is illustrated another embodiment of an elongated intervertebral implant 300 for replacement of a damaged spinal disk between adjacent vertebrae. The intervertebral implant 300 has a solid, block-like body 310 of any of the foregoing materials such as bone tissue, biocompatible metals, and biocompatible synthetic materials. The elongated intervertebral implant 300 includes a first surface 312, an opposing second surface 314, and a peripheral surface 318 extending there between. To provide the elongated shape to the body 310, the parallel first and second straight lateral edges 320, 322 are substantially longer than the third straight edge 324 extending between the first and second edges 320, 322. By elongating the shape the implant can be configured to assume the majority of the intervertebral space between even the largest or longest of the vertebrae, such as those associated with the lumbar region. Again, the intervertebral implant 300 can have a “D” shape provided by a curved fourth edge 326 located opposite the third straight edge 324 or, in other embodiments, can have other suitable shapes.

To retain the osteogenic or medicinal material, a plurality of apertures 330 can be disposed on non-perpendicular angles to the first surface 312 of the intervertebral implant 300. In particular, four separate apertures 330a, 330b, 330c, and 330d can be disposed along the elongated axis of the implant 300. The first two apertures 330a, 330b, are disposed near to the first lateral edge 312 and further are angled toward the first lateral edge. The second two apertures 330c, 330d are disposed near to the second side 314 and likewise are angled toward that second edge. Accordingly, in the illustrated embodiment the apertures 330 are not parallel to each other. As can be appreciated, all the plurality of apertures 330 could also be tapered, or only a portion of the plurality of apertures could be tapered.

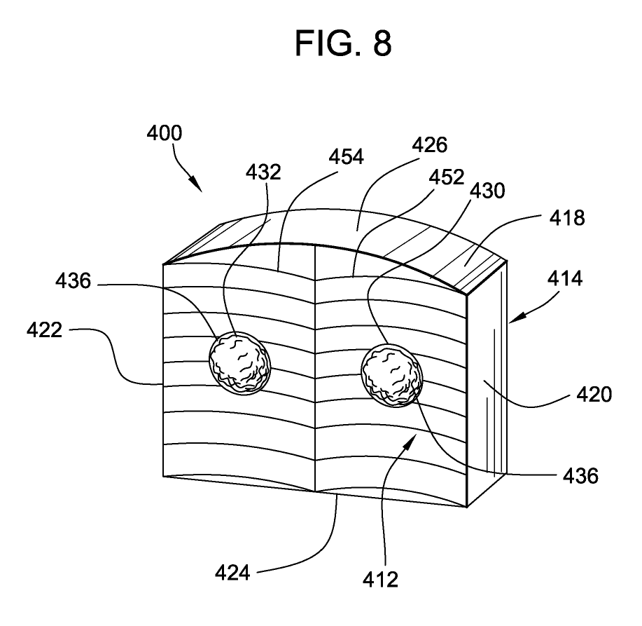

Referring to FIG. 8, in another aspect of the invention, the intervertebral implant 400 can include a plurality of grooves 450, as described below, disposed into one or more of its surfaces. The illustrated intervertebral implant 400 again can have a flat, block-like implant body 410 including a first surface 412 and an opposing second surface 414 interconnected by a peripheral surface 418. The peripheral surface can include a first straight lateral edge 420, a parallel second straight lateral edge 422, a third straight lateral edge 424 extending between the first and second edges 424, and a curved fourth edge 426 space apart and directed away from the third straight edge 424. The implant body can be made from any of the aforementioned suitable materials. Moreover, the intervertebral implant can include first and second apertures 430, 432 disposed therein that can be angled and/or tapered and as illustrated can retain osteogenic material 436.

In the illustrated embodiment, the plurality of grooves 450 are disposed across the first surface 412 and extend between the parallel first and second lateral edges 420, 422. In other embodiments, the grooves can be oriented in other directions to facilitate different insertion methods. The grooves can be disposed into the first surface any suitable depth, but should not thoroughly alter the strength or integrity of the intervertebral implant. For example, the depth of the grooves into the first surface can be about 1-2 mm and the spacing in between adjacent grooves in the plurality can be about 1 mm. Moreover, any number of grooves 450 can be in the plurality, and preferably the plurality of grooves are arranged in a gull-wing pattern. Specifically, the grooves 450 are parallel to each other and extend between the first and second adjacent lateral edges. To form the gull-wing pattern, the grooves 450 can each include a first curve 452 located proximate to the first lateral edge 420 and a second curve 454 located proximate to the second lateral edge 422. Both the first and second curves 452, 454 are directed towards the fourth curved edge of the implant, and the curves of each groove can intersect approximately mid-width between the first and second lateral edges. The grooves can help maintain position of the intervertebral implant sandwiched between the adjacent vertebrae by providing or encouraging friction between the surfaces of the implant and vertebrae that prevents slipping. In this regard, the gull-wing shaped grooves can be oriented so that the intersection between curves is in the direction of the intervertebral space into which the implant is inserted. This reduces the likelihood that the implant will become displaced before the implant and the vertebrae fuse together. Additionally, as illustrated, the plurality of grooves can also received and retain additional osteogentic material 436. Because the grooves extend across the surfaces 412, 414 of the implants 400, the osteogenic material is advantageously spread across the implant-vertebrae interface.

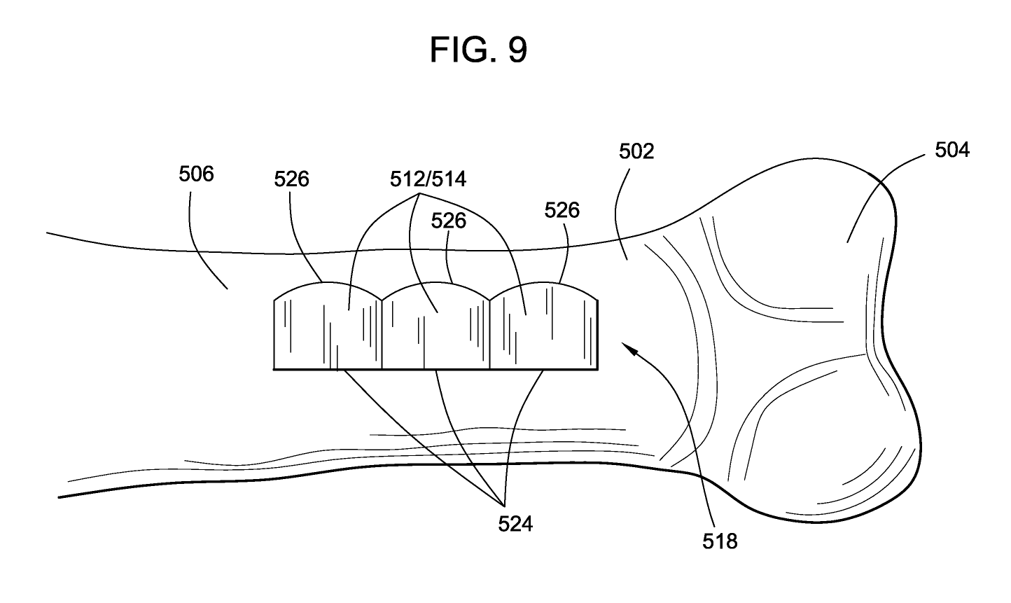

Described with respect to FIG. 9 is a method of producing intervertebral implants of the foregoing kind. In accordance with the method, there is provided a donor bone 502 from which the implants can be harvested. The donor providing the donor bone can be from the same or different species as the intended recipient of the implant. The donor bone 502 is preferably a longer bone such as a femur, tibia, or humerus. Accordingly, the donor bone 502 has a condyle 504 or rounded distal end supported on a diaphysis 506 or the narrower shaft of the bone. Cut into the diaphysis and spaced from the condyle 504 can be a plurality of outlines 518 that correspond to the peripheral surface of the intervertebral implants 500 to be formed. At this step, one of the opposing first and second major surfaces 512, 514 corresponds to the outer surface of the donor bone 502 while the other surface remains intact inside the bone. By cutting the outlines parallel to the axis of the diaphysis, the majority of the bone tissue in the intervertebral implant including that exposed on the first and second surfaces can be cortical bone tissue. The outlines 518 are cut such that a plurality of repeating outlines are linearly aligned along and parallel to the axis of the diaphysis 506. The plurality of outlines is removed from the donor bone 502 and can be separated from each other to provide the implant. To shape the flat body of the intervertebral implant, the portions of the outline corresponding to the first, second, and peripheral surfaces can be planed. Apertures and/or grooves of the foregoing type can be disposed into the implant and osteogenic material can be added. An advantage of cutting the implants from along the diaphysis is that doing so makes greater use of the surface area of the bone by allowing more outlines to be cut. Additionally, cutting parallel along the diaphysis allows implants of a larger and more varied shape to be produced. Furthermore, cutting from the diaphysis allows greater variability of the height of the implant measured between the third straight edge 524 and fourth curved edge 514, including enabling heights greater than 2.0 cm.

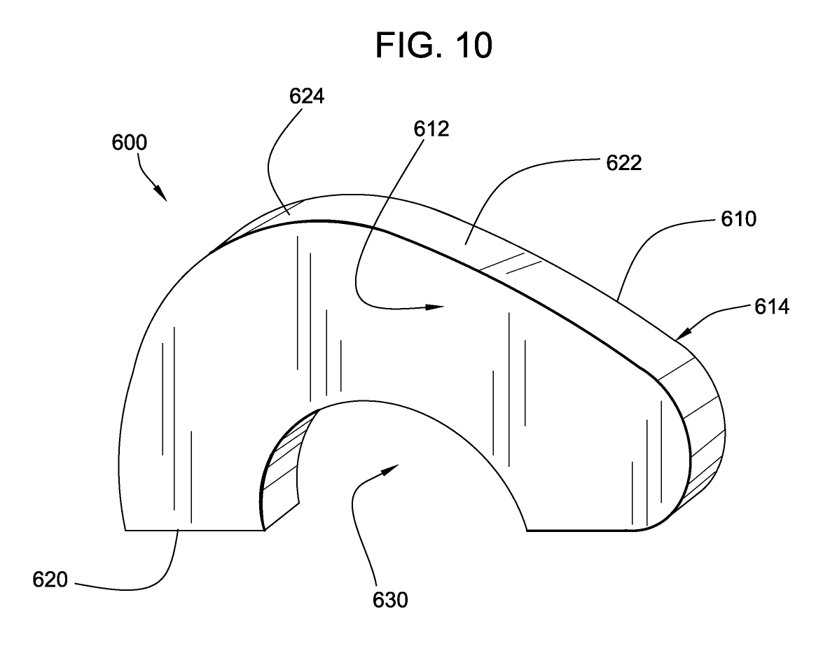

Referring to FIG. 10, there is illustrated another embodiment of an intervertebral implant 600 which is roughly shaped or outlined as a bow or question-mark. Particularly, the implant 600 can have a generally flat body 610 including a first surface 612, an opposing second surface 614, and a peripheral surface 618 interconnecting the first and second surfaces. To provide the bow or question-mark shape, the peripheral surface 618 can include a straight edge 620 and a curved edge 622 that bows outward and away from the straight edge. Moreover, the curved edge 620 can be distorted toward one half of the body 610. As illustrated, this causes the apex 624 of the curved edge 622 to be offset from the midpoint of the straight edge 620.

Disposed into the body 610 from the first straight edge toward the second curved edge 622 can be a cutout 630. The cutout 630 generally extends between and through the first and second surfaces 612, 614. Moreover, the cutout 630 can have any desired shaped and preferably has a rounded shape to conform generally to the shape of the curved edge 622. In various embodiments, the bone tissue proximate the curved edge 622 can be primarily cortical tissue while the bone tissue proximate the cutout 630 can be primarily cancellous tissue. The operation of forming the cutout 630 removes much of the cancellous tissue so that the remaining material of the implant 600 is primarily the biomechanically stronger cortical tissue. The cutout 630 can receive osteogenic material to promote bone growth and fusion of adjacent vertebrae. Moreover, in accordance with the foregoing embodiments, disposed into either or both of the first and second surfaces can be a plurality of grooves that can also prevent slipping of the inserted implant and/or receive osteogenic material.

The intervertebral implants described herein can be formed by any suitable forming operation. For example, a milling apparatus including a rotating end mill can be used to cut the implants from a donor bone and then to form the apertures and/or grooves. Additionally, the milling apparatus can be used with an end mill to plane the first and second surfaces so that the body is generally flat. To automate the process, the milling apparatus can be computer numerically controlled. In other embodiments, the intervertebral implants can be formed by traditional hand tools such as saw and/or osteotomes. After forming, the implants can be inserted freshly or can be stored in a frozen or freeze-dried state. In another embodiment, the intervertebral implant can have a feature that advantageously facilitates particular procedures for surgically inserting the implant between adjacent vertebrae. In this embodiment, the implant includes a shelve-like flange protruding from the main body that can be grasped by forceps or tongs during surgical implantation. Hence, the flange facilitates manipulation, orientation and placement of the implant into the intervertebral space by a surgeon during surgery.

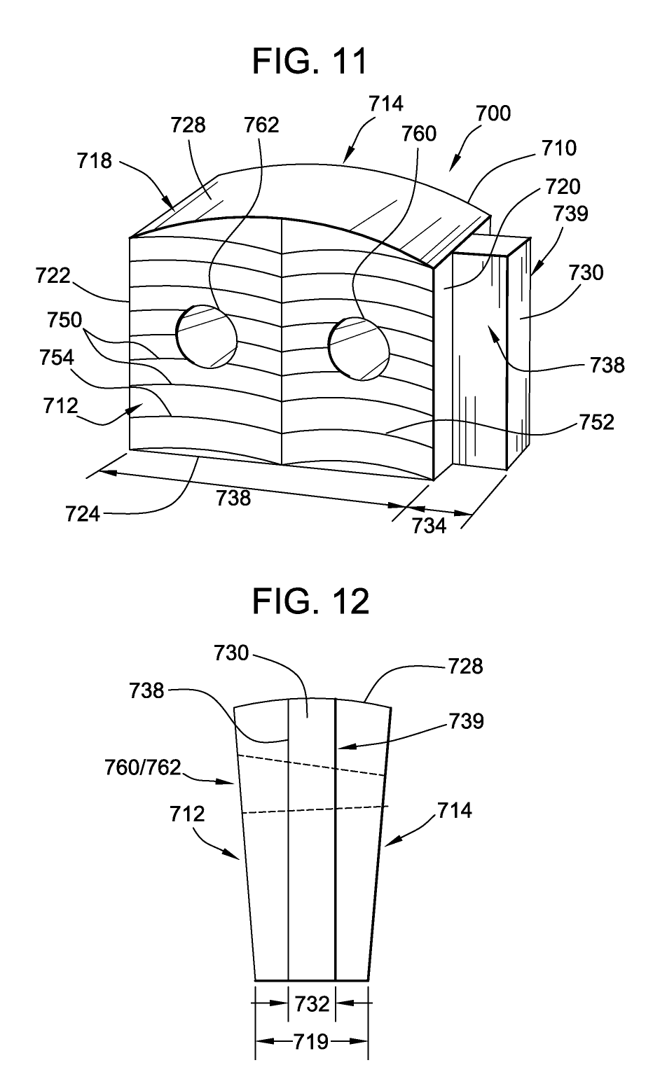

Referring to FIGS. 10 and 11, there is illustrated an embodiment of the intervertebral implant 700 having a flange 730. The implant includes a main body 710 that can have any suitable size and shape, but as illustrated has a flat, squat or puck-like shape. The flat shape is provided by a superior or upper first surface 712, an inferior or lower second surface 714 that is generally parallel and opposite the first surface, and a peripheral wall or surface 718 that extends between the upper and lower surfaces. The main body 710 is relatvely solid and can be made from any of the aforementioned materials to provide sufficient rigidity and load bearing characteristics for its intended application. The distance between the upper and lower surfaces 712, 714 delineates the thickness of the body 710, which is represented by arrow 719 in FIG. 11. The thickness 719 can be selected to correspond to the intervertebral space that the implant is intended for. In the particular embodiment, the main body 710 may be slightly inclined with the first and second surface 710, 712 slightly diverging away from each other but at such a minute degree that the body remains substantially flat and the surfaces remain substantially parallel.

The peripheral surface 718 delineates the outline of the main body 710 when the implant is viewed from above or below. In the particular embodiment, the outline is generally D-shaped but in other embodiments could have other suitable shapes such as oval or kidney shaped. The D-shaped outline of the main body 710 is conferred by a generally straight first lateral edge 720, a generally straight second lateral edge 722 and a rear edge 724 that extends between the first and second lateral edges. A curved front edge 728 opposite the rear edge 724 also extends between the first and second lateral edges 720, 722 and curves or is directed away from the rear edge so that the apex of the curved front edge is furthest from the rear edge. Because of the preferred process of cutting the implant 700 from a donor bone, it should be appreciated that the curved front edge 728 may not be a perfect or true curve but may be digitated from a series of smaller straight lines or edges. The degree or radius of curvature of the front edge 728, which may govern how far the apex of the front edge protrudes or is directed away from the rear edge 724, may vary from that illustrated in FIG. 11.

The flange 730 protrudes outwardly from the first lateral edge 720 of the main body 710 and is generally parallel with a plane defined by the first or second surfaces 710, 712. In the illustrated embodiment, the flange 730 is generally rectangular and block-like in shape and extends completely between the rear edge 724 and the curved front edge 728. The flange 730 itself may include a first flange surface 738 oriented toward the superior, first surface 712 of the main body 710 and a second flange surface 739 oriented toward the inferior, second surface 714.

However, the flange 730 is offset or spaced from both the first surface 712 and the second surface 714 such that the flange has a second thickness, represented by arrow 732, that is less than the first thickness. Hence, the flange 730 can be approximately half the thickness of the main body 710 of the implant 700. By way of example, if the thickness of the main body is about 8-14 millimeters, the thickness of the flange may be about 1-2 millimeter. Although offset from the first and second surfaces 712, 714, the rectangular flange 730 is generally parallel to the first and second surfaces and is located between imaginary planes defined by the first and second surfaces. The flange 730 is also oriented about mid-thickness of the main body 710 halfway between the first and second surface 712, 714. In other embodiments, the flange 730 may still have a thickness less than that of the main body 710, but can be co-planar with either the first or second surfaces 712, 714 so that when viewed from the side as shown in FIG. 11, the flange appears as a single step.

Additionally, the distance that the flange 730 protrudes from the main body 710, represented by arrow 734, can be any suitable distance but preferably should be sufficient to allow the flange to be grasped by forceps as described below. For example, if the width of the implant indicated by arrow 738 is 16 millimeters, the dimension 734 of the flange 730 can be 17 or 18 millimeters, or 1 to 2 millimeters of protrusion. The ratio of the thickness of the main body 710 to the width of the main body also demonstrates the flatness characteristic of the main body. For example, the ratio of width to thickness may be about 1:2, 1:3 or 1:4. Hence, the main body provides a relatively large surface area for the vertebrae to bear against, which thereby distributes the pressure forces transmitted through the implant more widely, while still being thin enough for insertion into the intervertebral space.

The flange can be formed integrally with the main body as part of the same block of material so that the flange and main block are unitary. As mentioned above, the flange and main body can be formed from any of the materials mentioned herein. In an embodiment, the flange and the main body can be formed from cortical bone material and/or cancellous bone material and can be cut together from the same donor bone. To make the spinal implant from a donor bone, it will be appreciated that an initial rough block of bone can be cut from the donor bone which can then be shaped by various carving techniques to produce the finished shape of the main body and flange. Additionally, the donor bone can be harvested by any suitable method from any suitable source including, preferably, the long diaphysis of the femur, tiba, or humerus as discussed with respect to FIG. 9.

The spinal implant 700 can include any of the features mentioned herein. For example, disposed on the first and/or second surfaces 712, 714 can be a plurality of grooves 750. The grooves 750 may traverse the implant 700 from the first lateral edge 720 to the second lateral edge 722. The grooves 750 can have any suitable shape or orientation, but in the illustrated embodiment the grooves 750 are gull-wing shaped. To form the gull-wing pattern, the grooves 750 include a first curve 752 extending from the first lateral edge 720 and a second curve 754 extending from the second lateral edge 722. The first curve 752 and the second curve 754 generally arc toward the front lateral edge 728 of the peripheral surface 718. The first and second curves 752, 754 can intersect approximately mid-width between the first and second lateral edges 720, 722 of the main body 710 of the implant. The gull-wing shaped grooves 750 can create friction between the upper and lower vertebrae reducing the likelihood that the spinal implant can shift, slide, rotate or otherwise move out of position before the vertebrae can fuse together. The forward or anterior orientation of the gull-wing shaped grooves 750, such that they arc or curve towards the front edge 728, simplifies insertion of the implant from the posterior toward the anterior of the spinal column.

Disposed into the implant 700 can be one or more apertures 760, 762, similar to the apertures described above. The apertures 760, 762 can be disposed into the first surface 712 and directed toward the second surface 714 and can either terminate just before the second surface or can break through the second surface. In the illustrated embodiment, the apertures 760, 762 are closed-ended and terminate approximately 1.0 millimeter before the inferior or second surface 714. In this embodiment, an osteogenic material for promoting bone growth and fusion of the vertebrae can be disposed or contained in the apertures 760, 762 without falling through the implant but can still promote vascular in-growth through the remaining 1.0 millimeter of bone implant material. The apertures as illustrated are circular but in other embodiments can include any suitable shape or cross-section. Additionally, the apertures 760, 762 can be disposed into the implant 700 at a non-perpendicular angle to the first surface 712, as described above. The apertures can have any suitable dimension, including a diameter of about 3 to 4 millimeters.

Disposed into the main body 710 of the implant and arranged in the gull-wing shaped grooves can be a plurality of micro-perforations 768, or small holes on the order of about 1.0 millimeter to about 0.1 millimeter in diameter. The micro-perforations 768 can be created by directing a sharp needle into the gull-wing shaped grooves 750 and pressing the needle into the material of the implant. The gull-wing shaped grooves 750 thereby layout the pattern for the micro-perforations and can help guide the needle into the implant material during their creation. The micro-perforations may facilitate vascular in-growth and new bone formation when the implant is situated in the spinal column.

Like the grooves and apertures discussed above, the micro-perforations 768 can contain or include an osteogenic material to promote bone growth and fusion of the vertebrae. Any of the suitable, aforementioned osteogenic materials can be applied to the implant. In one embodiment, the osteogenic material can be a particulate bone material such as that described in U.S. Pat. No. 7,335,381, issued on Feb. 26, 2008 and assigned to Losec, Inc. of Houston, Tex., which is hereby incorporated by reference in its entirety. That patent describes both a bone composition and a method of preparing the bone composition that has desirable particulate size ranges and is prepared under conditions that promote osteoinductive properties of the bone. For example, the particulate sizes of the bone material is preferably 355 μm or less and the particulate can be ground from solid bone material under conditions that substantially prevent the temperature of the bone and grinder from rising above 33° C. It has been found that these conditions are beneficial to preserving the osteogenic properties of the particulate material. The disclosed material can be included on the intervertebral spinal implant via any of the aforementioned manners.

Referring to FIG. 12, there is illustrated a method of replacing a disk with the intervertabral implant. A surgeon can use a pair of forceps 790 to grasp the implant by the flange 730 such that the forceps bear upon the first and second flange surfaces 738, 739 as shown. The surgeon can align the implant 700 between an upper vertebra 780 and an adjacent lower vertebra 782 that were separated by the disk that is intended to be replaced. The flange 730 provides an easy-to-grasp surface for the surgeon to place the forceps 790 on. The implant 700 can be aligned so that the curved forward edge 728 is directed toward the anterior of the spinal column and the rear edge 724 is directed toward the posterior of the spinal column. The surgeon then inserts the implant into the intervertebral space between the upper and lower vertebrae 780, 782. The superior or first surface 712 of the implant 700 can be oriented toward the upper vertebra 780 and the inferior or second surface 714 can be oriented toward the lower vertebra 782. The squat shape of the implant and the relative large surface areas provided by the first and second surface 712, 714 facilitate the load bearing function of the implant after insertion and the transmission of weight forces through the spinal column. Because the flange 730 is offset from the main body 710, the surgeon does not have to grasp the main body of the implant reducing the likelihood that the forceps could interfere with the implantation procedure.

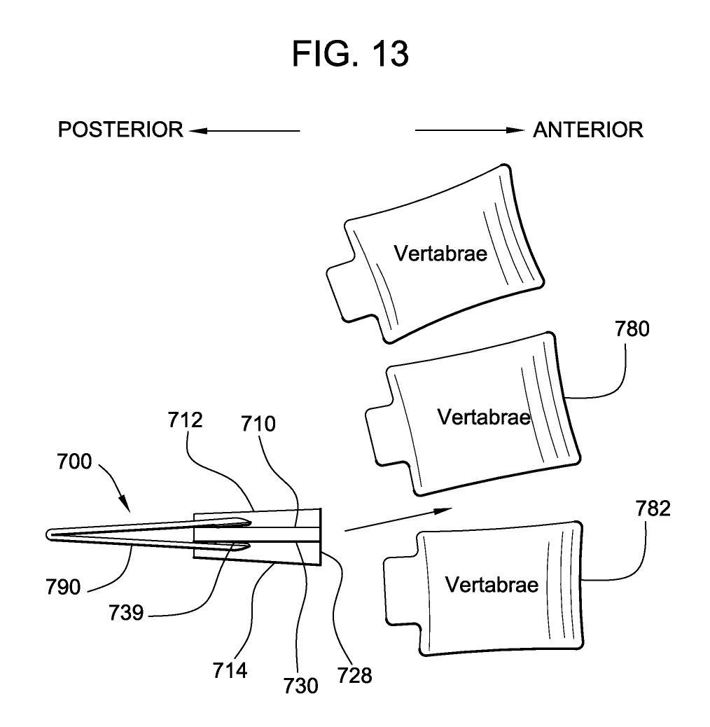

For example, referring to FIG. 13, the implant 700 is illustrated inserted between the upper and lower vertebrae 780, 782 with the main body 710 substantially inline with the spinal column as represented by arrow 784. The flange 730 however lies outside the arrow 734 delineated by the spinal column, such that it can be easily grasped by a surgeon using a pair of forceps in a manner such that the forceps will not interfere with placement of the implant in the intervertabral space. Moreover, because of the reduced thickness of the flange 730 compared to the main body 710 of the implant 700, the protruding flange 730 remains spaced apart and thus freely suspended from the inferior or lower surface of the upper vertebra 780 and from the superior or upper surface of the lower vertebra 782. That spacing or gap between the flange 730 and the vertebrae allows the surgeon to grasp and manipulate the spinal implant with the forceps even when the first surface of the main body is in adjacent contact with the upper vertebra 780 and the second surface is in adjacent contact with the lower vertebra 782. Likewise, the surgeon can easily release the flange 730 with the forceps and remove the forceps from the incision during surgery. In other embodiments, it will be appreciated that the flange can be freely suspended within the columnar outline of the spinal column but can, due to its offset design and the resulting spacing from the upper and lower vertebrae, it can still be accessed with the forceps. The upper vertebra 780 can completely overlap the first surface 712 of the main body 710 of the spinal implant and the lower vertebra 782 can completely overlap the second surface 714 of the main body. Once the spinal implant 700 is situated in the intervertebral space between the upper and lower vertebrae, the implant will provide rigid biomechanical support and stability for the spinal column and can promote bone growth causing the upper and lower vertebrae to fuse together.

Although in FIG. 13 a single implant is illustrated as being inserted into the intervertebral space between the adjacent vertebrae, in other embodiments multiple implants can be sized and shaped to fit into the intervertebral space. For example, the thickness of the implants can be less than that discussed above so that multiple implants can be stacked together vertically in the intervertebral space between upper and lower vertebrae. One advantage of this procedure is that it allows greater flexibility in that the surgeon can select the appropriate number of implants to be used to replace differently sized disks along the spinal column.

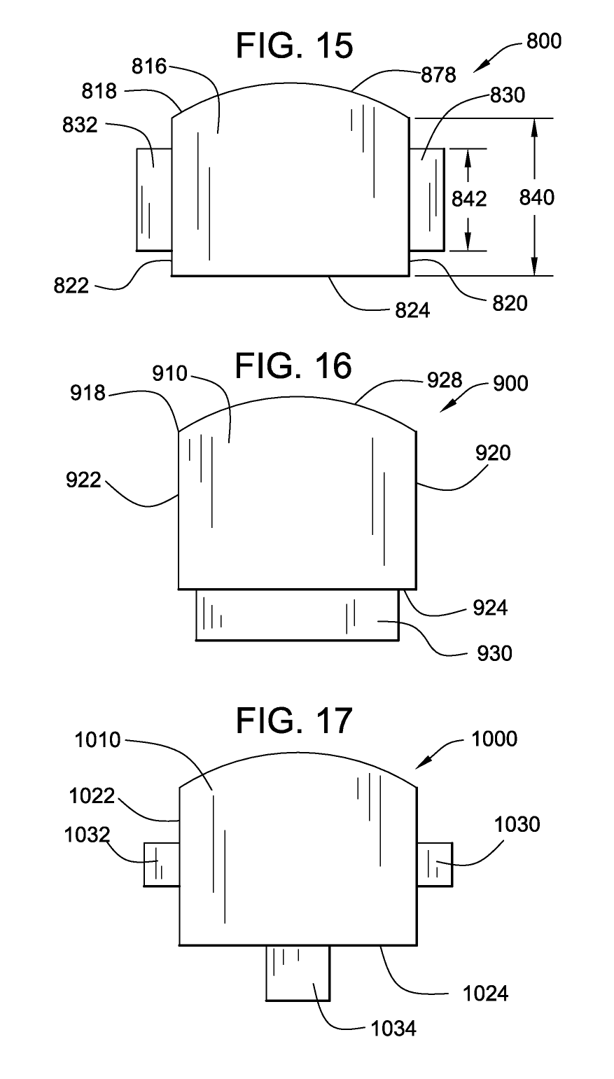

Referring to FIG. 15, there is illustrated another embodiment of the spinal implant 800 which includes a first flange 830 and a second flange 832 protruding from the main body 810. Specifically, the illustrated implant is generally D-shaped and the peripheral surface 818 can include a first lateral edge 820, a generally parallel second lateral edge 822, a generally straight rear edge 824 that extends between the first and second lateral edges and a curved forward edge 828. Protruding from the first lateral edge 820 is the first flange 830 and protruding from the second lateral edge 822 is the second flange 832. As described above, the first and second flanges 830, 832 can have a thickness dimension that is less than that of the implant. An advantage of providing first and second flanges 830, 832 is that the surgeon can grasp the implant from either side with the forceps during surgery thereby providing greater flexibility during the implantation procedure.

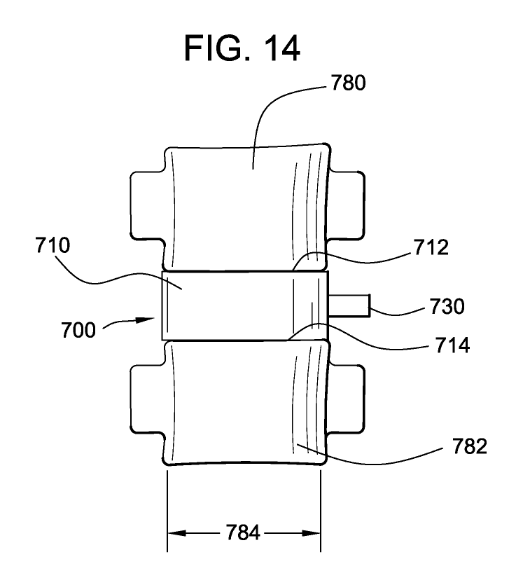

FIG. 15 also illustrates another possible feature of the flanges 830, 832 in that they may be shorter than the lateral edges of the implant. For example, the first lateral edge 820 may have a length indicated by arrow 840 of about 16 millimeters, while the first flange 830 may have a length indicated by arrow 842 of about 10 to 15 millimeters. Because the first flange 830 is shorter than the first lateral edge 820, it can be offset or set back from the intersection of the first lateral edge and the rear edge 824. The first flange can also be offset from the intersection of the first lateral edge 820 and the curved front edge 828. Although the embodiment of the spinal implant having two flanges illustrated in FIG. 14 has smooth, planar surfaces with no grooves or apertures disposed into the surfaces, it will be appreciated that the embodiment could readily include either or both of these features.

Referring to FIG. 16, there is illustrated another embodiment of a spinal implant 900 having a flange 930 extending from the main body 910. The implant 900 can be generally flat and has a D-shaped outline as defined by its peripheral surface 918. To provide the D-shaped outline, the peripheral surface 918 includes a generally straight first lateral edge 920, a parallel second lateral edge 922, a rear edge 924 extending between the first and second lateral edges, and a curved forward edge 928 opposite the rear edge. The flange 930 protrudes rearwardly from the rear edge 924 and can be grasped with a pair of forceps during the insertion procedure. It should be appreciated that when the spinal implant 930 is situated in the intervertebral space, the flange is directed toward the posterior of the body. An advantage of having the flange 930 face the rearward or posterior direction is that the flange is less likely to interfere with any muscles or organs that are located along the sides of the spinal column.

Another possible advantage may be realized during the surgical procedure illustrated in FIG. 12. Because the surgeon can grip the posterior-directed flange 930 with the forceps when inserting the implant toward the anterior of the spinal column, the forceps do not need to enter the intervertebral space between the vertebrae. This is advantageous in part because during posterior transplants the dural covering over the spinal cord must be retracted to expose the intervertebral space. Even when the dural covering has been cut open and retracted though, the dural covering restricts access to the intervertebral space limiting the room in which the surgeon has to manipulate the implant and instruments. The flange 930 can be sized so that when the implant is placed between adjacent vertebrae, the implant and the flange can be substantially overlapped by the upper and lower vertebrae. The flange is therefore in a substantially covered or protected position where it will minimize interference with muscles or other organs along the lateral sides of the spinal column. In other embodiments, it will be appreciated that the insert may be inserted via laparoscopic surgical techniques that will avoid any anterior or posterior exposure of the intervertebral space and allow for preservation, at least in part, of the anterior and/or posterior ligaments.

Referring to FIG. 17, there is illustrated another embodiment of an intervertebral spinal implant 1000 having a plurality of flanges protruding from the main body 1010. By way of example, the implant is again substantially D-shaped and its peripheral edge 1018 can include a generally straight, first lateral edge 1020, a generally straight, parallel second lateral edge 1022, and a generally straight rear edge 1024 that extends between the first and second lateral edges. Disposed toward the front of the implant 1000 is a front curved edge 1028. In the illustrated embodiment, a flange is disposed on and protrudes from each of the generally straight lateral edges and the generally straight rear edge. Hence, the implant 1000 includes a first flange 1030 on the first lateral edge 1020, a second flange 1032 on the second edge 1022 and a third flange 1034 on the rear edge 1024 which protrudes in the rearward direction. The flanges may be substantially shorter than the lateral or rear edges but should still be large enough to allow them to be easily grasped with a pair of forceps. An advantage of providing multiple flanges is that it allows the implant to be grasped from either side or from the rear during implantation hence allowing for greater flexibility during the insertion procedure.

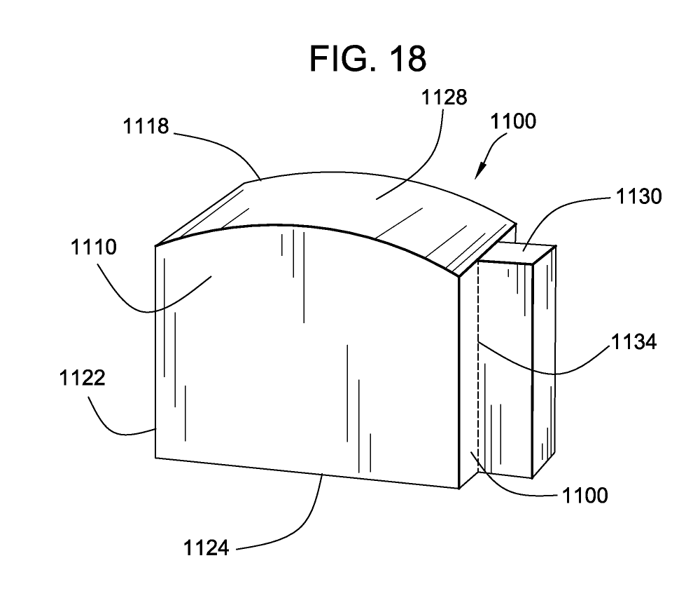

Referring to FIG. 18, there is illustrated another embodiment of a spinal implant 1100 having a flange 1130 protruding outwardly from the main body 1110. The main body 1110 of the spinal implant 1100 is illustrated has having a D-shaped outline as delineated by the peripheral surface 1118 and specifically provided by a generally straight, first lateral edge 1120, a parallel second lateral edge 1122, a posterior-directed rear edge 1124 and a curved front edge 1128. The flange 1130 protrudes from the first lateral edge 1120. In the illustrated embodiment, rather than being formed integrally with the main body 1110, the flange 1130 can be formed separately and attached to the first lateral edge 1120 as indicated by dashed line 1134. To attach the flange 1130 to the main body 1110, an adhesive or complementary form-locking features such as a tongue-and-groove structure can be used. In another possible variation, the flange 1130 may be attached to the main body 1110 in such a way that it can be detached by the surgeon during the surgical procedure. For example, a shallow groove can be made along the line indicated by 1134 on one or both sides of the flange 1130 that will allow the flange to break off from the main body 1110. After the main body 1110 is inserted and sandwiched between the adjacent vertebrae, the flange can be detached and removed so that it no longer protrudes outwardly from the spinal column in a manner that could interfere with or injure muscles or other organs of the patient.

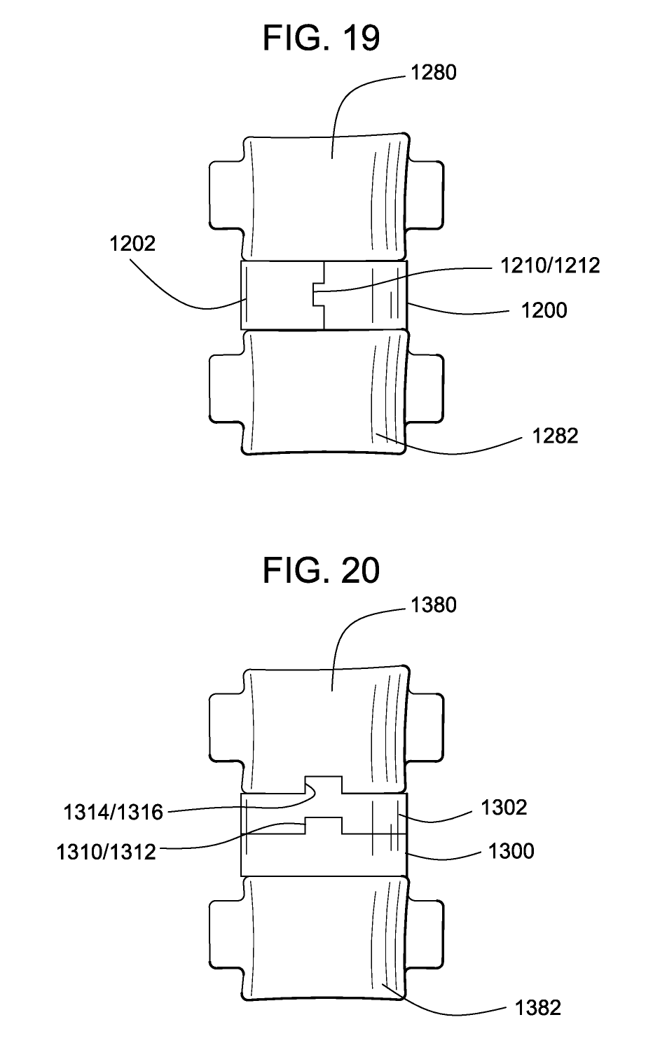

Referring to FIG. 19, there is illustrated another embodiment of the spinal implant 1200 having a tongue-and-groove design to facilitate interoperation with a corresponding second implant 1202. For example, the first and second implants 1200, 1202 are situated laterally side-by-side in the intervertebral space between an upper vertebra 1280 and lower vertebra 1282. The implants 1200, 1202 are sized so that when placed side-by-side they both can fit adjacently within the columnar area between the upper and lower vertebrae. To help the implants maintain their position between the vertebrae, the first implant 1200 can include a protruding tongue 1210 extending from a lateral edge of the implant that can be received in a corresponding groove 1212 disposed into a lateral edge of the second implant. Hence, when the first and second implants are placed side-by-side in the appropriate orientation, they can generally interlock with each other.

Referring to FIG. 20, there is illustrated another embodiment of the spinal implants having a tongue and groove design to help prevent the implants from becoming dislocated before the vertebrae have a chance to fuse together. In the illustrated embodiment, the first implant 1300 and the second implant 1302 are stacked vertically in the intervertebral space between the upper vertebra 1380 and the lower vertebra 1382. Although only two implants are illustrated, any suitable number of implants can be used as is necessary to fill the intervertebral gap between the vertebrae. To help maintain the positioning of the first and second implant, the first implant 1300 can include a tongue 1310 protruding upwards from its superior or upper surface that can be received in a corresponding groove 1312 disposed along the inferior or lower surface of the second implant. The tongue and groove 1310, 1312 are illustrated as extending from the posterior toward the anterior of the spinal column, but in other embodiments, could extend in different directions.

A further feature illustrated in FIG. 20 is a second protruding tongue 1314 disposed along the upper surface of the second implant 1302 that can be received in a corresponding groove 1316 disposed into the upper vertebra 1380. To create the groove 1316 in the upper vertebra, the surgeon will have to cut away or remove bone material from the vertebra during the surgical procedure. The interconnecting tongue 1314 and groove 1316 between the second implant 1302 and the upper vertebra 1380 helps prevent the implant from shifting or dislocating and may help promote bone growth and fusion.

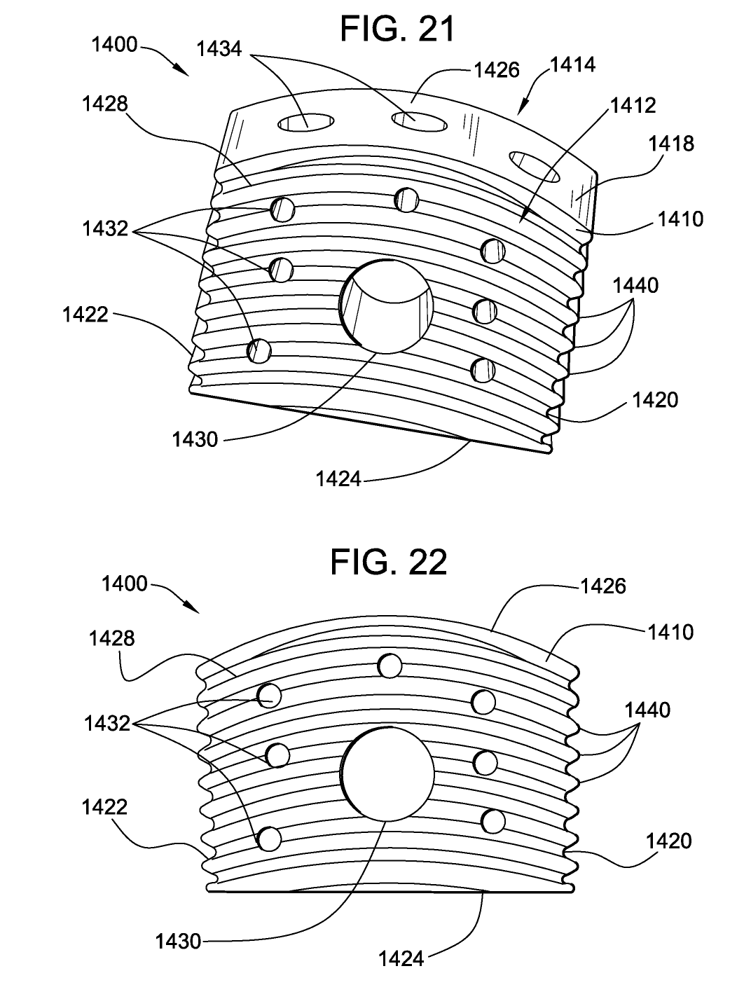

Referring to FIGS. 21 and 22, there is illustrated another embodiment of the spinal implant 1400 having a plurality of apertures disposed into it. More particularly, the implant 1400 includes a main body 1410, preferably of bone material, which is generally flat and rectangular in shape and includes a first surface 1412 and a generally parallel, opposing second surface 1404 that are joined by a peripheral surface 1418. To delineate the generally rectangular shape or outline of the main body 1410, the peripheral surface 1418 can include a first lateral edge 1420, a second lateral edge 1422 parallel to and spaced apart from the first lateral edge, and a rear edge 1424 that extends between the first and second lateral edges. The main body 1410 can also include a forward front edge 1426 that is slightly curved or bowed outwardly and that is opposite and directed away from the rear edge 1424. In addition to some of the other embodiments described herein, the first and/or second surface 1412, 1414 can have a plurality of curved grooves 1428 disposed therein.

In the illustrated embodiment, there is disposed into the main body 1410 a plurality of apertures including a centrally located main aperture 1430 and a plurality of smaller apertures or micro-perforations 1432 that are located about the main aperture. As illustrated in FIGS. 21 and 22, the main aperture 1430 can be located generally equidistant between the first and second lateral edges 1420, 1422 and generally equidistant between the front and rear edges 1424, 1426. The main aperture 1430 and micro-perforations 1432 are disposed from the first surface 1412 through to the second surface 1414, but in other embodiments may only be partially disposed into the main body 1410. The micro-perforations may have any suitable size, and preferably have a diameter of about 1 millimeter. In addition to being disposed into the first surface 1412, in the illustrated embodiment, a second set of micro-perforations 1434 can also be disposed into the curved front edge 1426. As discussed above, both the main aperture 1430 and the micro-perforations 1432 can include osteogenic material. In some embodiments, the number of micro-perforations and their size can be configured and varied to allow surgeons and medical practitioners to vary the amount of osteogenic material disposed in the micro-perforations in discrete measurements. For example, the plurality of micro-perforations can have different sizes or depths to hold 5 mm³, 10 mm³, 15 mm³, etc. of osteogenic material. Another possible advantage of the micro-perforations is that they promote fusion by providing voids in which bone growth from adjacent vertebra may be accommodated.

In another variation illustrated in the embodiments of FIGS. 21 and 22, the first and second lateral edges 1420, 1422 can have a corrugated or wrinkled form or structure. To provide the corrugated or wrinkled pattern, each lateral side edge 1420, 1422 can include a series of linear ridges or corrugations 1440 that extend from the first surface 1410 to the second surface 1412. One possible advantage of the corrugated edges is that the implant will be easier to grasp with a pair of tongs or forceps during implantation.

Hence, the present disclosure provides an intervertebral spinal implant that can replace an injured spinal disk to provide support to the spinal column and promote adjacent vertebrae to fuse together. The spinal implant can include a flange protruding from its peripheral or median surface that enables a surgeon to grasp, manipulate and orientate the implant during the insertion procedure. Because the flange protrudes from the main body of the implant, it simplifies use of forceps during insertion of the implant between adjacent vertebrae. In other embodiments, rather than replace a damaged disk, the implant can replace an actual vertebra of the spinal column. In this version, the implant can serve as a vertebral body replacement part.

All references, including publications, patent applications, and patents, cited herein are hereby incorporated by reference to the same extent as if each reference were individually and specifically indicated to be incorporated by reference and were set forth in its entirety herein.

Terms such as “upper,” “lower,” “superior,” “inferior,” “front,” “rear,” “anterior,” “posterior,” and the like are for reference purposed only and are not intended as a limitation on the claims in any way. The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An intervertebral spinal implant for insertion between adjacent vertebra comprising:
a generally flat, substantially solid body of bone material having a first surface, a generally parallel opposite second surface, the first surface and second surface defining a first thickness of the body, a peripheral surface between the first an second surfaces delineating an outline of the body; and
a shelve-like flange protruding from the peripheral surface configured for grasping with forceps, the flange having a second thickness less than the first thickness.

2. The intervertebral implant of claim 1; wherein the flange has a first flange surface and a second flange surface, the first and second flange surfaces generally parallel to the first and second surface of the body.

3. The intervertebral implant of claim 2, wherein the outline of the body is generally D-shaped, and the peripheral surface includes a generally straight, first lateral edge, a generally straight, second lateral edge generally parallel to the first lateral edge, a generally straight rear edge extending between the first and second lateral edges, and a curved front edge extending between the first and second lateral edges and curving away from the third edge.

4. The intervertebral implant of claim 3, wherein the flange extends from the first lateral edge.

5. The intervertebral implant of claim 4, wherein the flange extends from the rear edge away from the front edge.

6. The intervertebral implant of claim 4, wherein the body has a first length along the first lateral edge between the rear edge and the front edge, and the flange has a second length that is less than the first length.

7. The intervertebral implant of claim 6, wherein the flange is intermediate and offset from the rear edge and the front edge.

8. The intervertebral implant of claim 3, wherein the flange extends from the rear edge.

9. The intervertebral implant of claim 1, further comprising a second flange protruding from the peripheral surface.

10. The intervertebral implant of claim 1, wherein the body includes one or more grooves disposed into and traversing at least one of the first and second surfaces between the first and second lateral edges.

11. The intervertebral implant of claim 1, wherein grooves are gull-wing shaped each including a first and second curves directed toward the curved front edge of the body and intersecting approximately mid-width between the first and second lateral edges.

12. The intervertebral implant of claim 1, further comprising at least one aperture disposed into the body from the first surface toward the second surface.

13. The intervertebral implant of claim 1, further comprising an osteogenic material applied to the implant.

14. The intervertebral implant of claim 13, wherein the osteogenic material comprises particulate bone including particles having sizes less than or equal to about 355 .mu.m and having a particle size distribution including from about 24.6 wt % to about 36.3 wt % of particles having a particle size between about 350 .mu.m and about 250 .mu.m, from about 22 wt % to about 25 wt % of particles having a particle size between 250 .mu.m and about 150 .mu.m, and from about 36.7 wt % to about 46.7 wt % of particles having a particle size less than 150 .mu.m, and prepared from bone having an initial temperature between about 18.degree. C. and about 20.degree. C. and ground in a mill under conditions so that the bone is not heated above a critical temperature of less than or equal to 40.degree. C., where the particulate bone is non-chemically extracted, non-demineralized, and where said composition has improved osteoinductive activity and regeneration of bone defects as compared to demineralized particulate bone.

15. The intervertebral implant of claim 1, wherein the body and the flange are unitary.

16. The intervertebral implant of claim 1, wherein the flange is attached to the body.

17. A method of surgically inserting an intervertabral implant into an intervertebral space between two adjacent vertebra to promote fusion of the vertebra, the method comprising:
(i) providing an intervertebral implant comprising a generally flat body having a first surface, a second surface generally parallel and opposed to the first surface, a peripheral wall between the first and second surfaces, and a flange protruding from the peripheral surface;
(ii) grasping the flange with a pair of forceps;
(iii) inserting the implant between the vertebra; and
(iv) releasing the forceps from the implant.

18. The method of claim 15, wherein after insertion, the first surface of the body adjacently contacts the upper vertebra and the second surface adjacently contacts the lower vertebra.

19. The method of claim 16, wherein the upper and lower vertebra completely overlap the body.

20. An intervertebral spinal implant for insertion between adjacent vertebra comprising:
a generally flat, substantially solid body of bone material having a first surface, a generally parallel opposite second surface, the first surface and second surface defining a first thickness of the body, a peripheral surface between the first an second surfaces delineating an a generally rectangular outline of the body;
a centrally located aperture disposed through the first surface into the body of bone material; and
a plurality of micro-perforations
